(19) United States
(12) Patent Application Publication
(10) Pub. No.: US 2026/0055003 A1
(43) Pub. Date: Feb. 26, 2026

(54) METHOD FOR SYNTHESIZING A CALCIUM-BASED BIOMINERAL UNDER AMBIENT CONDITIONS

(71) Applicant: City University of Hong Kong, Hong Kong (HK)

(72) Inventors: Xinxue TANG, Hong Kong (HK); Chong WANG, Hong Kong (HK); Junda SHEN, Hong Kong (HK); Zhengtao XU, Hong Kong (HK); Jian LU, Hong Kong (HK); Yang Yang LI, Hong Kong (HK)

(21) Appl. No.: 18/813,070

(22) Filed: Aug. 23, 2024

(51) Int. Cl. C01F 11/18; A61K 6/20; A61K 6/864; A61K 6/873; C01B 25/32

(52) U.S. Cl. CPC C01F 11/185; A61K 6/20; A61K 6/864; A61K 6/873; C01B 25/32; C01P 2002/01; C01P 2002/74

(57) ABSTRACT

The present invention relates to a method for synthesizing calcium-based biominerals, particularly hydroxyapatite (found in vertebrate bones and teeth) and calcium carbonate (found in plankton and corals). This fully biocompatible mineralization strategy not only offer fundamental insights into the biomineralization process, starting from mineral ions to amorphous precursors and eventually to specific minerals, but also contributes to our scientific understanding of life's origin. Furthermore, it showcases great promise for therapeutic applications, such as actively repairing damaged tooth enamel and promoting bone growth.

### FIELD OF THE INVENTION

The present invention generally relates to at least the fields of dental materials and orthopedic materials manufacturing, as well as material synthesis and processing, and so forth.

### BACKGROUND OF THE INVENTION

Biominerals, such as calcium orthophosphates (e.g., hydroxyapatite, Ca₁₀(PO₄)₆(OH)₂) and calcium carbonates (e.g., calcite, CaCO₃), form essential skeletal components in organisms. Biomineralization occurs diversely across microorganisms, marine creatures, and humans. Depending on conditions such as pressure, acidity, temperature, and surrounding substances, a wide variety of biominerals can be selectively formed. Among them, hydroxyapatite (HAP) and calcium carbonate are crucial for both vertebrates and invertebrates.

Biominerals in organisms are formed from disordered precursor phases, in which the crystallization behaviors can be precisely regulated under physiological conditions¹⁻³. Current studies on biomineralization precursors often focus on amorphous powders or polymer-induced liquid precursors (PILPs) derived from amorphous calcium carbonate (ACC). Many important discoveries have been made, such as the hydrated nature of the various amorphous precursors⁴, the effects of Mg²⁺ and other cationic additives on the stability and properties of ACC⁵⁻⁶, the influence of the organic chaperons on the mineralization behaviors of ACC¹⁻², the role of hydration water in the fusion of ACC particles or mineral gels under compression, and biomineralization-inspired enamel repair⁷⁻⁸.

Despite these exciting achievements, biomineralization remains full of mysteries. One major puzzle involves the initiation and control of the crystallization of amorphous precursors under mild aqueous conditions. Another mystery concerns the origin of biominerals, specifically whether different calcium-based biominerals share the same origin.

Besides their structural function, biominerals play crucial roles in various diseases. For example, the deposition of dihydrate calcium pyrophosphate (Ca₂P₂O₇·2H₂O) has been linked to the onset of acute arthritis known as Calcium Pyrophosphate Deposition (CPPD)⁹. Beyond its pathological implications, pyrophosphate (P₂O₇⁴⁻), the shortest polyphosphate, plays a crucial role in energy transfer and utilization within living organisms. It is essential in the physiological synthesis of proteins, RNA, DNA, and other biomolecules, and is continuously produced by the enzyme pyrophosphatase in living cells. However, preparing pyrophosphates under biological, non-enzymatic conditions in the laboratory is challenging, typically requiring high temperatures of around 600-1300° C.

Commercial production of HAP often faces significant synthetic difficulties in controlling the particle size, morphology, as well as requiring complicated equipment. Similarly, the commercial synthesis methods of CaCO₃, CaK₃H(PO₄)₂, Na₂Ca(CO₃)₂·5H₂O and Na₃Mg(PO₄)(CO₃) are limited to direct chemical synthesis by mixing ionic solutions. The commercial methods for producing Ca₂O₂P₇·2H₂O also presents notable synthetic difficulties. This invention addresses these needs.

### SUMMARY OF THE INVENTION

The present invention aims to eliminate the need for traditionally harsh experimental conditions to get the unique biominerals.

In particular, the present invention provides a cost-effective, mild and biocompatible method for synthesizing a calcium-based biominerals, including mixing one or more biocompatible aqueous solutions containing more than three cationic species and/or anionic species to form a stable and non-toxic amorphous starting gel; and transferring the stable and non-toxic amorphous starting gel into one or more salt solutions to trigger phase transformation to form the calcium-based biominerals. The one or more salt solutions are adopted in different concentrations.

In one embodiment, the method further includes washing and drying the stable and non-toxic amorphous starting gel. The drying can be achieved through air-drying or freeze-drying.

In one embodiment, the stable and non-toxic amorphous starting gel remains in an amorphous state and does not crystallize when exposed to temperatures below 600° C.

In one embodiment, the calcium-based biomineral includes calcium orthophosphates, calcium carbonates, phosphate carbonates, or calcium pyrophosphates.

In one embodiment, the one or more biocompatible aqueous solutions include calcium chloride solution, magnesium chloride solution, sodium carbonate solution, dipotassium phosphate solution, or a combination thereof.

In one embodiment, the cationic species are selected from the group consisting of calcium, strontium, magnesium, manganese, iron, cobalt, nickel, copper, zinc, silver, gold, ammonium, sodium, potassium cations, and rare earth elements.

In one embodiment, the anionic species are selected from the group consisting of molybdate, tungstate, sulfate, silicate, phosphate, chloride, and carbonate anions.

In one embodiment, the biocompatible aqueous solutions used to prepare the amorphous starting gel have concentrations ranging from 0.1 M to 1M.

In one embodiment, the one or more salt solution includes CaCl₂ solution, K₂HPO₄ solution, or Na₂CO₃ solution.

In one embodiment, the stable and non-toxic amorphous starting gels are mixed with the salt solution in concentrations ranging from 0.4 M to saturation.

In one embodiment, the calcium-based biominerals have different average particle size ranging from 1 nm to 700 nm.

In another aspect, the present invention provides a bone cement material containing said calcium-based biomineral.

In another aspect, the present invention provides a tooth repair material containing said calcium-based biomineral.

The method for fabricating biominerals of the present invention offers several advantages: (1) the biominerals can be produced from a stable and non-toxic amorphous multiple-ionic gel; (2) the experimental setup is simple, requiring at most an oven, freeze-drying machine, and centrifuge, without the need for expensive equipment such as vacuum systems, protective gases, or sophisticated control systems typically required by other technologies; (3) this innovative process demonstrates its potential as an effective method for producing hydroxyapatite. These findings contribute to the advancement of biomaterials research and hold promise for improving dental care, bone-related interventions, and standard experimental procedures for the production of commercial chemicals.

### BRIEF DESCRIPTION OF THE DRAWINGS

Embodiments of the invention are described in more details hereinafter with reference to the drawings, in which:

FIG. 1A illustrates a synthesis process for the amorphous starting gel. FIG. 1B depicts plots of G′ (storage modulus) and G″ (loss modulus) versus shear frequency for the amorphous starting gel (25° C., 40% humidity). FIG. 1C depicts XRD patterns of the amorphous starting gel after stored for 12 hrs and 120 days (22-25° C., approximately 40% humidity). FIG. 1D shows TEM SAED images and EDS elemental mapping of the amorphous starting gel;

FIG. 2A depicts XRD patterns and FTIR spectra of “+Ca-dry-HAP” obtained with different soaking time of 10, 120, and 480 min and finally dried at 60° C. FIG. 2B depicts XRD patterns of “+Ca-dry-HAP”, “+P-dry-HAP”, and “+C-dry-CaC”, fabricated by soaking the amorphous starting gel in different concentrations of CaCl₂) (0.40 to 6.7 M), K₂HPO₄ (0.40 to 13.44 M), and Na₂CO₃ (0.40 to 1.93 M). The soaking time for all the samples was 8 hrs. FIG. 2C depicts XRD patterns of “gel+Ca” and “+Ca-frzdry-CPP” prepared with different soaking time (10, 120, and 480 min) in the CaCl₂ solution (6.7 M). FIG. 2D depicts Raman spectra of “gel+Ca” and the amorphous starting gel. FIG. 2E shows a photograph showing ample CO₂ bubbles generated with “+Ca-frzdry-CPP” (prepared via soaking in 6.7 M CaCl₂ solution for 480 min) dissolved in a solution of HCl (15 wt %);

FIG. 3 shows a schematic illustration of Ca²⁺-triggered mineralization pathway;

FIG. 4A depicts XRD patterns of the stable and non-toxic amorphous gel and the products of “gel+Ca”, “+Ca-frzdry-CPP”, and “+Ca-dry-HAP” (prepared with a soaking time of 480 min; “+Ca-dry-HAP” dried at 60° C.). FIG. 4B shows TEM images, SAED diffraction, and EDX elemental mapping of “+Ca-frzdry-CPP” shown in FIG. 4A;

FIG. 5A shows TEM images, SAED pattern, and EDX elemental mapping and composition of “+Ca-dry-HAP” that is prepared with a soaking time of 480 min). FIG. 5B depicts elemental atomic contents (vs. that of P) for the stable and non-toxic amorphous gel and “+Ca-dry-HAP” (prepared with a soaking time of 480 min) characterized by ICP;

FIG. 6A shows photograph and SEM image of the amorphous starting gel. FIG. 6B depicts EDS spectra of the amorphous starting gel. FIG. 6C shows elemental mapping for Ca, Mg, C, P of the amorphous starting gel;

FIG. 7A shows photograph and SEM image of the “+Ca-dry-HAP” (prepared via soaking in 6.7 M CaCl₂ solution for 480 min). FIG. 7B depicts EDS spectra of the “+Ca-dry-HAP”. FIG. 7C shows elemental mapping for Ca, Mg, C, P of the “+Ca-dry-HAP”;

FIG. 8A depicts atomic ratios of Mg:P of the amorphous starting gel, “+Ca-dry-HAP”, and “+Ca-spnt”. FIG. 8B depicts atomic contents of the different elements in “+P-spnt” and “+C-spnt”;

FIG. 9 depicts time-evolving XRD patterns of the wet “ACCP” gel collected after stored under ambient conditions for different time durations;

FIG. 10A depicts microhardness and modulus values of amorphous starting gel and “+Ca-dry-HAP” (prepared via soaking in 6.7 M CaCl₂ solution for 480 min). FIG. 10B shows a photograph of “+Ca-dry-HAP”. FIG. 10C shows SEM images of amorphous starting gel and “+Ca-dry-HAP”;

FIG. 11 depicts XRD patterns of the “+Ca-frzdry-CPP” (prepared via soaking in 6.7 M CaCl₂ solution for 480 min) sample as freeze-dried and after thermally annealed at different temperatures (450 and 600° C.) for 2 hours;

FIG. 12A shows a schematic illustration of HPO₄²⁻-triggered mineralization pathway. FIG. 12B depicts XRD patterns of the “+P-dry-HAP” samples soaked for different time. Samples are dried in air at 60° C. FIG. 12C depicts XRD patterns of the “+P-dry-HAP” samples produced with different soaking times in the concentrated K₂HPO₄ solution (13.44 M);

FIG. 13 shows a schematic illustration of CO₃²⁻-triggered mineralization pathway;

FIGS. 14A-14C show XRD patterns of the mineralization products obtained with different soaking solutions of varying pH values (all soaked for 20 hrs, washed with DI water, and air-dried at 60° C.);

FIG. 15A depicts cell viability of hBMSCs cultured with DMEM containing different concentrations of amorphous starting gel for 72 hours. FIG. 15B shows H&E histological staining images of hBMSCs cultured with DMEM containing different concentrations of amorphous starting gel for 72 hours. FIG. 15C depicts ALP activities of hBMSCs cultured with blank DMEM (negative control), ODM (positive control), and ODM+amorphous starting gel for 4, 7 and 10 days. FIG. 15D shows ALP staining images of hBMSCs cultured with blank DMEM (negative control), ODM (positive control), and ODM+amorphous starting gel for 4, 7 and 10 days. FIG. 15E depicts ARS absorbance quantitative analysis of hBMSCs cultured with blank DMEM (negative control), ODM (positive control) and ODM+amorphous starting gel for 21 days; FIG. 15F depicts ARS staining images of hBMSCs cultured with blank DMEM (negative control), ODM (positive control) and ODM+amorphous starting gel for 21 days. The statistical significance with p<0.01 and p<0.001 in comparison with the positive control is indicated by ** and *** in FIG. 15C and FIG. 15E;

FIG. 16 shows photographs of the native tooth before and after the acid etching (etched for 10 sec);

FIG. 17 shows a schematic illustration of the enamel repair procedure; and

FIG. 18A depicts modulus and hardness of the original tooth enamel, the etched enamel (soaked in 37% H₃PO₄ solution for 10 s), and the regenerated enamel. FIG. 18B depicts nanoindentation curves for the native, acid-etched, and recovered enamels. FIG. 18C shows SEM images for H₃PO₄-etched enamel and the enamel repaired with epitaxial growth of HAP.

FIG. 18D shows fluorescent photos and confocal images of the regenerated enamel treated with calcein-labeled calcium ion solution to highlight the newly grown Ca-containing layer.

### DETAILED DESCRIPTION

In the following description, methods for fabricating biominerals including calcite (CaCO₃), hydroxyapatite, or the dihydrate calcium pyrophosphate (Ca₂P₂O₇·2H₂O) are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The existing mineralized amorphous precursors are difficult to preserve in physical temperature and humid environments and will quickly transform into a more stable and low-energy crystalline state. For instance, the commercial methods for producing HAP often face significant synthetic difficulties in controlling the particle size, morphology, and complicated equipment requirements. The commercial methods for producing CaCO₃, CaK₃H(PO₄)₂, Na₂Ca(CO₃)₂·5H₂O and Na₃Mg(PO₄)(CO₃) are limited in direct chemical synthesis by mixing ionic solutions. The commercial methods for producing Ca₂O₂P₇·2H₂O often presents synthetic difficulties. Normally, the crystallized Ca₂O₂P₇·2H₂O will be synthesized by the hydrothermal procedure at 900-1300° C.

According, in a first aspect, the present invention provides a cost-effective, mild and biocompatible method for synthesizing a calcium-based biominerals, including mixing one or more biocompatible aqueous solutions containing more than three cationic species and/or anionic species to form a stable and non-toxic amorphous starting gel; and transferring the stable and non-toxic amorphous starting gel into one or more salt solution to trigger phase transformation to form the calcium-based biomineral.

The one or more biocompatible aqueous solutions are mixed in the same concentration and volume. For example, if there are “n” different ionic species, the concentrations and volume ratios of the “n” solutions are each 1, where “n” is the number of different solutions used.

By using the present method, the biominerals, particularly calcite and hydroxyapatite, can thus be easily obtained in a biocompatible manner. Additionally, the present invention can also readily produce dihydrate calcium pyrophosphate, contrasting other laboratory synthesis of pyrophosphate that requires high temperature.

In one embodiment, the calcium-based biomineral may include calcium orthophosphates, calcium carbonates, phosphate carbonates, or dihydrate calcium pyrophosphate (Ca₂P₂O₇·2H₂O). Examples of the calcium orthophosphates include hydroxyapatite, Ca₁₀(PO₄)₆(OH)₂, calcium phosphate, or CaK₃H(PO₄) 2. Examples of the calcium carbonates include CaCO₃, calcite, vaterite, aragonite, Na₂Ca(CO₃)₂, or ikaite. Examples of the phosphate carbonates include Na₃Mg(PO₄)(CO₃).

Not limited to this, other calcium-based biominerals may also be formed, such as brushite (CaHPO₄·2H₂O), octacalcium phosphate (OCP, Ca₈(H₂PO₄)₆·5H₂O), tricalcium phosphate (TCP, Ca₃(PO₄)₂), monocalcium phosphate (MCP, Ca(H₂PO₄)₂), fluorapatite (Ca₁₀(PO₄)₆F₂).

The calcium-based biomineral may exhibit various structures, including but not limited to spherical, rod-shaped, and plate-like forms.

The calcium-based biominerals may be homologous, meaning they can both originate from the same precursor. Specifically, the precursor is a stable and non-toxic amorphous gel. The extremely stable starting gel features an amorphous framework of mesoporous nanoparticles with uniform elemental distribution.

The starting gel is a stable and non-toxic amorphous gel easily prepared by mixing common water-soluble inorganic salts. In one embodiment, the stable and non-toxic amorphous starting gels are synthesized from biocompatible aqueous solutions containing typical ions found in organisms (e.g., Ca²⁺, Mg²⁺, CO₃²⁻, and HPO₄²⁻).

In one embodiment, the aqueous solutions may include, but are not limited to CaCl₂), MgCl₂, Na₂CO₃, and K₂HPO₄ solutions.

The stable gel-like state is likely due to the “high entropy” generated by multiple ionic components and various water species. Kinetically, the complex mixture maintains the gel-like amorphous state through spatial hindrance, viscosity, or internal stress. Thermodynamically, the high entropy decreases free energy of the amorphous gel, while the multiple ionic and hydration species set space hindrances and barriers for kinetic structural rearrangement, both preventing crystallization.

To enable the high entropy, it is crucial to involve more than 3 types of coprecipitating ions. For example, the following simpler mixtures, with only three coprecipitating ions, do not form as stable amorphous gels: Gel 1 (by mixing Mg²⁺, Ca²⁺, and CO₃²⁻ ions), Gel 2 (by mixing Mg²⁺, Ca²⁺, and PO₄³⁻ ions), and Gel 3 (by mixing Ca²⁺, CO₃²⁻, and PO₄³⁻ ions). These products all crystallized within 7 day.

In this invention, the stable and non-toxic amorphous starting gels can endure high temperatures without crystallizing, remaining in an amorphous state up to temperatures below 600° C.

Ions triggered crystallization can be reliably triggered by dispersion in salt solutions, evolving into a wide variety of biominerals. When dispersed in a Na₂CO₃ or K₂HPO₄ solution, the multi-ionic, “supervariate” gel can selectively form the biomineral calcite (CaCO₃) or hydroxyapatite, respectively. More notable reactions occur when the gel is dispersed in a CaCl₂) solution under ambient conditions, resulting in the formation of a less common biomineral, dihydrate calcium pyrophosphate (Ca₂P₂O₇·2H₂O, CPP), within the morphologically intact gel matrix. The resulting gel further transforms into hydroxyapatite upon drying at room temperature.

The synthesized calcium-based biominerals has been successfully explored in dental recovery treatments, expanding the range of materials available for tooth protection and repair. Additionally, they enrich the selection and methods of bone materials, rendering it valuable for various applications in bone tissue engineering and restorative procedures.

### EXAMPLE

### Example 1—Materials and Methods

### Materials

The chemicals were purchased without further purification, such as CaCl₂) (purity ≥97%, Sigma-Aldrich), MgCl₂ (purity ≥95%, Sigma-Aldrich), Na₂CO₃ (purity ≥99.5%, Sigma-Aldrich), K₂HPO₄ (purity ≥98%, Sigma-Aldrich), H₃PO₄ (purity ≈85%, Sigma-Aldrich), Calcein (purity ≥95%, Sigma-Aldrich), HCl (purity=35-37%, Duksan). Human teeth were provided by the Baocheng Hospital (Shenzhen) in this invention. The use of human tooth tissue specimens followed a protocol that was approved by the ethical committee of the hospital and the City University of Hong Kong and agreed upon by the patients.

### Characterization

X-ray diffraction (XRD) patterns were collected on an X-ray powder diffractometer (BRUKER SRD-D2 Phaser) using Cu-Kα radiation (λ=1.5406 Å) (scanning rate of 10°/min). The Rheometer used was Kinexus pro⁺ with a test gap of 1 mm, a frequency range of 0.1-100 Hz, and the oscillatory mode (22-25° C., 40-45%). Transmission electron microscope (TEM) images were collected on FEI Talos F200S (FEI Super-X EDS Detector). The scanning electron microscope (SEM) images and spectra were obtained using a field-emission SEM (Philips XL-30) equipped with an energy-dispersive X-ray (EDX) detector operating at 20 kV. Nanoindentation tests were collected on a G200 nanoindentation instrument (Agilent Technologies, CA, USA). A constant strain rate of 0.2 nm s 1 was maintained during the loading process. The applied load force and the depth of continuous penetration into the sample during indentation were monitored by a computer. Microindentation was tested on the Fischer HM2000XY Micro-Hardness Tester. Fourier-transform infrared spectroscopy (FTIR) data were collected from a PerkinElmer FTIR Spectrometer. Raman spectra were collected from the WITec RAMAN alpha 300R.

### Confocal Imaging

The Ca²⁺ in the soaking solution was labelled by calcein. First, the untreated portion was coated with nail polish as a control for comparison. After the repair test, the enamel was studied under confocal microscope. Fluorescence images were taken using an inverted confocal laser scanning microscope (Nikon-Eclipse 90i) under illumination with a 488 nm laser. All images were analysed using image analysis software (NIS-Elements).

### General Cell Culture

Bone marrow mesenchymal stem cells (BMSCs) were cultured in low glucose Dulbecco's Modified Eagle Medium (DMEM, Invitrogen), supplied with 10% heat-inactivated fetal calf serum (Invitrogen) and 1% antibiotic-antimycotic agent (Invitrogen), in an atmosphere of 5% CO₂ at 37° C. The culture medium was substituted every three days, and the cell confluence was kept under 85%.

### Cell Viability Assay

Cytotoxicity was investigated via Cell Counting Kit-8 (CCK-8) test. The BMSCs were seeded in a 96-well plate (1×10⁴ each well) for 24 hours with DMEM. The DMEM was then removed, refilled with a DMEM solution mixed with the gel of the present invention (e.g., stable and non-toxic amorphous starting gel (wt. % of the freeze-dried gel: 1, 2, or 5 mg/mL)), and incubated for another 72 hours. A blank DMEM was used as control. CCK-solution (10 μL) was then added to each well of the plate, and further incubated for another 120 min. The absorption at the wavelength of 450 nm was measured on a microplate reader (Beckman Coulter DTX 880).

### Cell Morphology and Density Evaluation

Cell morphology and density evaluation were conducted via hematoxylin and eosin staining (H&E staining). The BMSCs were incubated in a DMEM solution mixed with the stable and non-toxic amorphous starting gels (wt. % of the freeze-dried gel: 1, 2, or 5 mg/mL) for 72 hours. A blank DMEM was used as control. The cells were fixed in 10% formalin solution for 20 min and stained by soaking in the hematoxylin solution for 3 min, and then rinsed by water for 1 min. The sample were then differentiated in 1% acid ethyl alcohol and blued in 0.2% ammonia solution for 15 sec. Finally, the samples were stained with eosin solution for 60 sec and dehydrated through an ascending concentration of ethyl alcohol. The photographs were taken using an optical microscope (Leica DMI3000).

### Biomineralization Assay

Alizarin red S (ARS) staining was conducted to investigate the biomineralization of the stable and non-toxic amorphous starting gels. In this experiment, 3 groups were set up, including a negative control group, a positive control group and an experimental group. For the negative control group, the BMSCs were cultured with normal DMEM. For the positive control group, the BMSCs were cultured with osteogenic differentiation medium (ODM) which was composed of normal DMEM, 50 μg/mL ascorbic acid (Sigma, USA), 10 mM β-glycerol phosphate (MP Biomedicals, France) and 100 nM dexamethasone (Santa Cruz, UK). For the experimental group, the BMSCs were cultured with ODM supplied with 5 mg/mL stable and non-toxic amorphous starting gels. The BMSCs were seeded in a 24-well dish with a 1×10⁵ cells/cm² density and cultured with normal DMEM for all the groups first.

When the confluence reached 80%, each group was refreshed with the corresponding culture medium. The osteogenesis capability of the stable and non-toxic amorphous starting gels was examined by ARS staining. Observing and capturing images were performed on an optical microscope (Leica DMI3000). Quantification analysis was performed by dissolving the stained samples in a solution of 70% methanol and 10% acetic acid in distilled water. The absorption of 450 nm was measured via a microplate reader (Beckman Coulter DTX 880). The quantitative assay was triplicated to avoid contingency. The data was analyzed through a t-test to examine the result's significance (p value).

### Osteogenic Assay

Alkaline phosphatase (ALP) assay was applied to characterize the osteogenic effects of the stable and non-toxic amorphous starting gels. Three groups (negative control, positive control, and the stable and non-toxic amorphous starting gel) were tested. For the negative control group, the BMSCs were cultured with the DMEM. For the positive control group, the BMSCs were cultured with osteogenic differentiation medium (ODM) which was composed of DMEM, 50 μg/mL ascorbic acid (Sigma, USA), 10 mM β-glycerol phosphate (MP Biomedicals, France), and 100 nM dexamethasone (Santa Cruz, UK). For the stable and non-toxic amorphous starting gels sample, the BMSCs were cultured with ODM supplied with 5 mg/mL stable and non-toxic amorphous starting gels.

The BMSCs were seeded in a 24-well dish at a density of 1×10⁵ cells/cm² and cultured with DMEM first. When the confluence reached 80%, each group was refreshed with the corresponding culture medium. On Day 4, 7, and 10, the cells were stained with ALP staining solution (Beyotime, China). The photographs were taken on an optical microscope (Leica DMI3000).

Meanwhile, the cells were lysed for conducting ALP activity assay (Beyotime, China). The absorbance was measured through a microplate reader (Beckman Coulter DTX 880) under the illumination with the wavelength of 405 nm. The quantitative assay was triplicated to avoid contingency. The data was analyzed through a t-test to examine the result's significance (p value).

### Example 2

### Preparation of the ACCP Gel and Stable and Non-Toxic Amorphous Starting Gels

The Na₂CO₃ and K₂HPO₄ solutions (each 50 mL, 0.8 M) were mixed in Beaker A (capacity 500 mL); the CaCl₂ solutions (100 mL, 0.8 M) were placed in Beaker B (capacity 500 mL). Under vigorous magnetic stirring, the solutions in Beaker A and Beaker B were quickly mixed (within a few seconds), followed by further stirring at ca 1300 r/min for 15 min, during which the clear mixture gradually turned white. The supernatant thereof was removed by centrifuge and decanting; and the obtained gel mass was washed by being dispersed in DI water and then centrifuged and decanted. The washing treatment was repeated three times. The resulting gelatinous substance was denoted as “ACCP” gel.

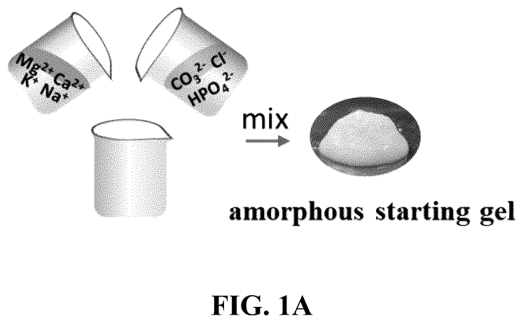

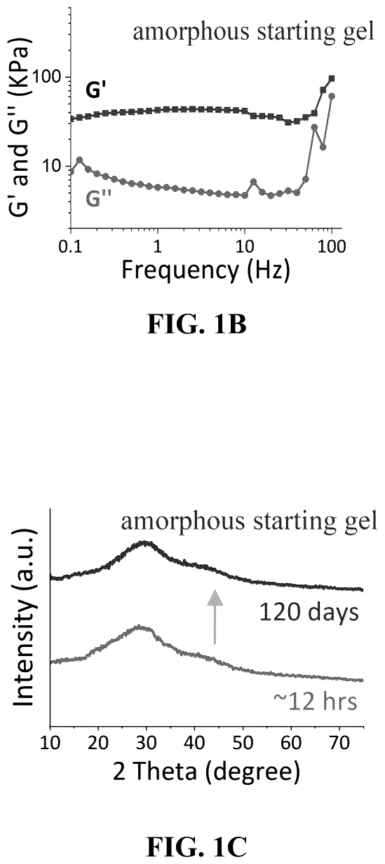

Four stock solutions of CaCl₂), MgCl₂, Na₂CO₃, and K₂HPO₄ (each of 0.8 M) were prepared. As shown in FIG. 1A, the Na₂CO₃ and K₂HPO₄ solutions (each 50 ml) were mixed in Beaker A (capacity 500 ml); the CaCl₂) and MgCl₂ solutions (each 50 ml) were mixed in Beaker B (capacity 500 ml). Under vigorous magnetic stirring, the solutions in Beaker A and Beaker B were quickly mixed (within a few seconds), followed by further stirring at ca 1300 r/min for 15 min, during which the clear mixture gradually turned white. The supernatant thereof was removed by centrifuge and decanting; and the obtained gel mass was washed by being dispersed in DI water and then centrifuged and decanted. The washing treatment was repeated at least three times. All experiments were performed at room temperature (22-25° C.).

Optionally, other additive ions other than Ca²⁺, Mg²⁺, CO₃²⁻, and PO₄³⁻ could also be added.

The ultra stable amorphous starting gel appeared milky white to the naked eye. FIGS. 1A-1B confirmed its colloidal, and gelatinous nature with even main elemental distribution of Ca, Mg, C, and P in the mineral moiety (FIG. 1D). The gel exhibited high resistance to crystallization for at least 120 days under ambient conditions (wet, 22-25° C.) (FIG. 1C).

### Example 3

### Preparation of Biominerals

By immersion in different ionic solutions, the stable and non-toxic amorphous starting gels gradually transformed into various biominerals, including calcium carbonates (CaCO₃), hydroxyapatite, and pyrophosphate (Ca₂P₂O₇·2H₂O). This innovative mineralization approach showed significant promise for diverse applications, especially in dental repair and osteogenesis.

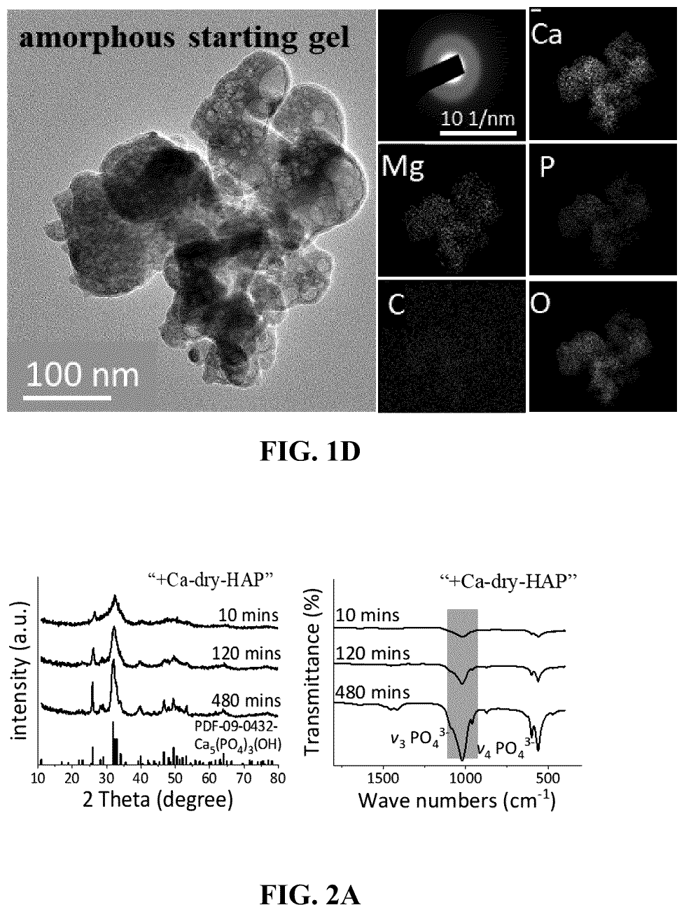

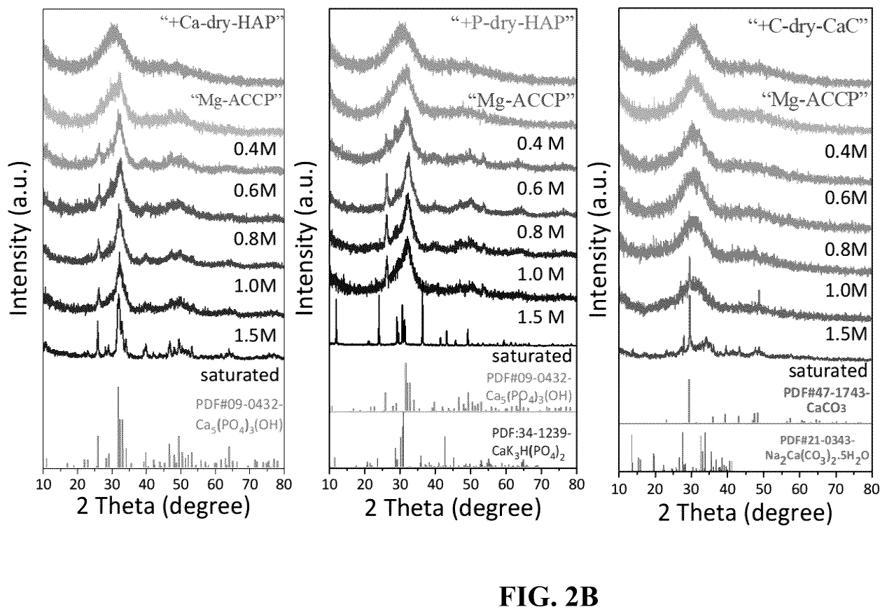

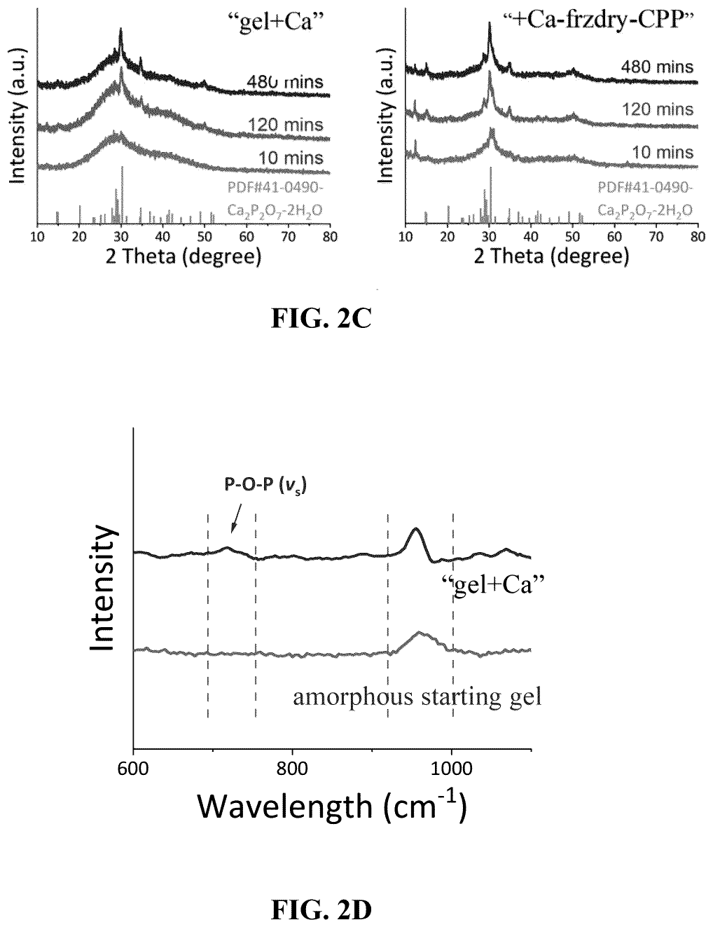

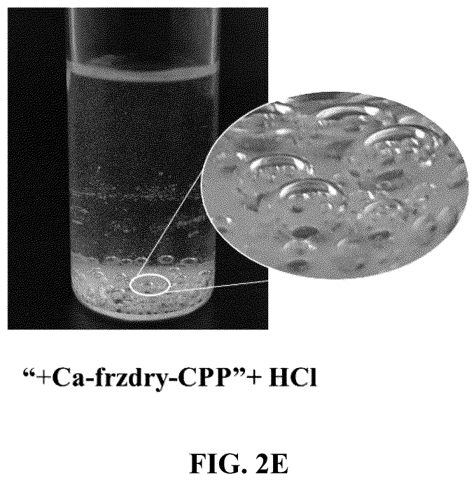

Referring to FIGS. 2A-2E, the ion-triggered mineralization process commenced rapidly, with crystalline XRD peaks observed within 10 minutes of immersion. The process continued over time and could be expedited by increasing the concentration of the soaking solution.

### Preparation of the “Gel+Ca”, “+Ca-Spnt”, “+Ca-Frzdry-CPP”, and “+Ca-Dry-HAP” Samples

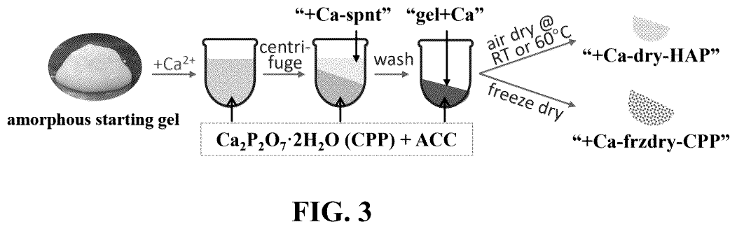

Referring to FIG. 3, the wet stable and non-toxic amorphous starting gel (15 g) from Example 2 was dispersed in 30 ml solution of CaCl₂) (0.4, 0.6, 0.8, 1.0, 1.5, or 6.7 M) under the Vortex agitation. After standing for 15 hours, the white suspension was centrifuged. The supernatant collected from the sample treated with 6.7 M CaCl₂ solution was collected and labeled as “+Ca-spnt”. The precipitates were washed three times (by being redispersed in DI water, centrifuged, and decanted) to remove the soluble ions. After draining the supernatant, the precipitate thus obtained was a gelatinous substance, denoted as “gel+Ca”. The subsequently freeze-dried sample was named “+Ca-frzdry-CPP” and those air-dried at 60° C. or at room temperature were named “+Ca-dry-HAP”.

The Ca²⁺-triggered mineralization of the stable and non-toxic amorphous starting gel was ascribed to the following reaction mechanism. First, with the excess Ca²⁺ ions in the environment, the Mg²⁺ ions in the gel were substituted and dissolved, producing calcium pyrophosphate and amorphous calcium carbonate (ACC) which was possibly stabilized by impurities (such as phosphates). When air dried under low heat (e.g., 60° C.) or room temperature, pyrophosphate further reacted with ACC to produce crystalline “+Ca-dry-HAP”:

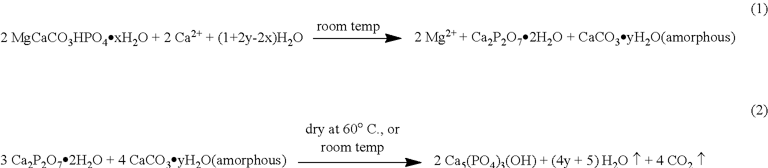

The ion-triggered mineralization described here introduces a mild and convenient biocompatible approach to produce unconventional minerals such as pyrophosphate, which are not easily accessible through conventional methods. Given the critical roles of pyrophosphates in key physiological processes (including their presence in synovial fluid and association with CPPD disease or ‘pseudogout’), the inorganic and fully biocompatible mineralization discovered here provides insights into the origins of pyrophosphates on prebiotic Earth and their involvement in the pathogenic pathways of ‘pseudogout’.

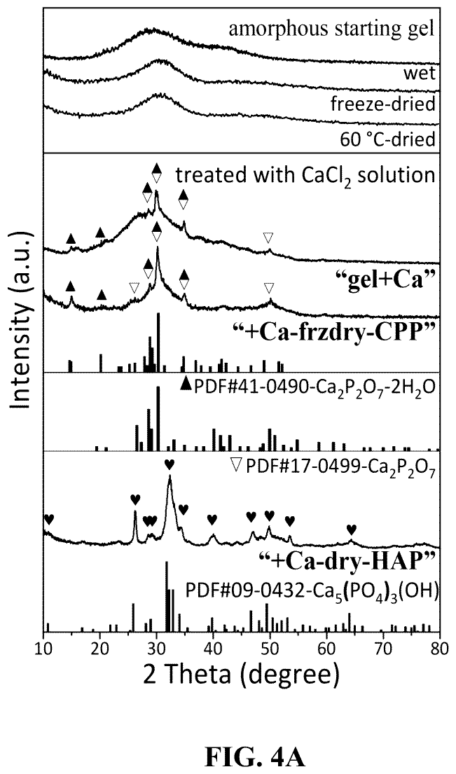

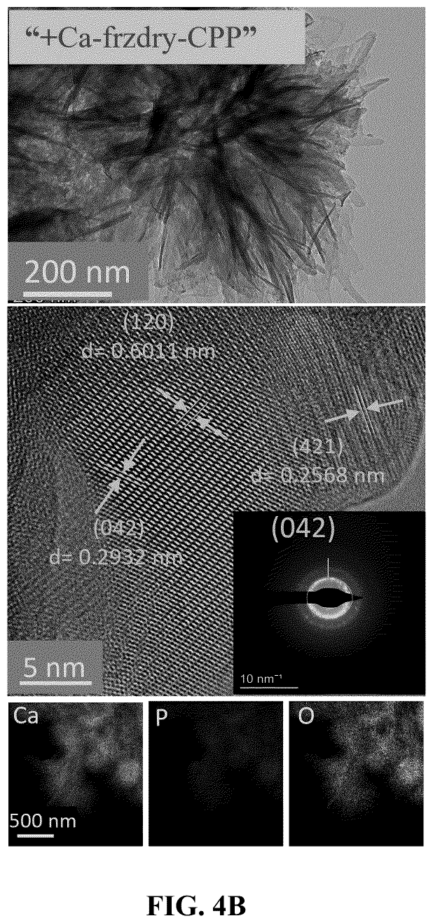

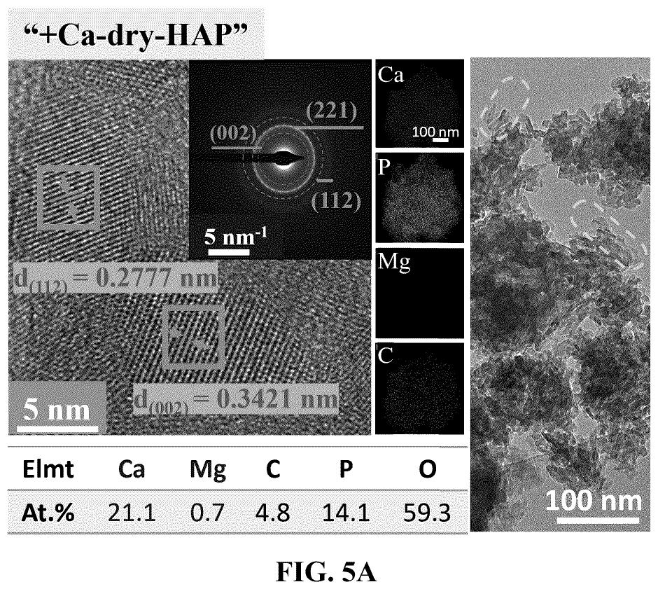

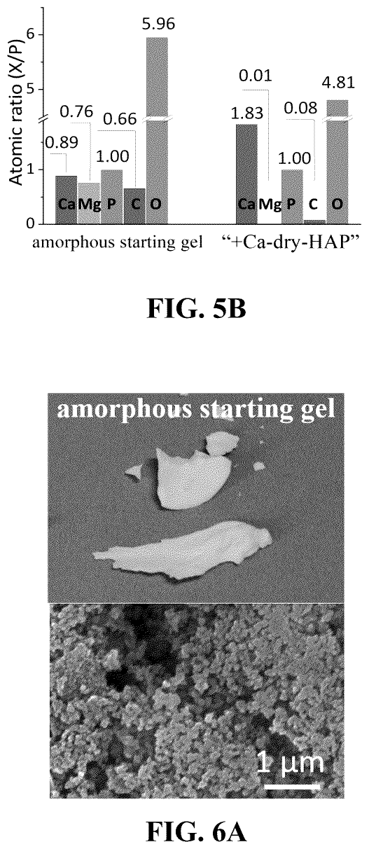

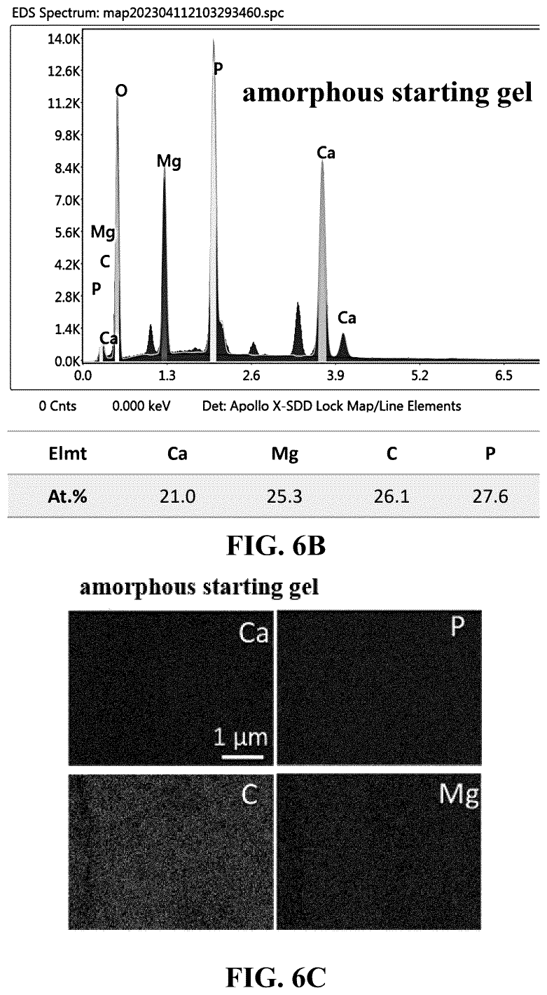

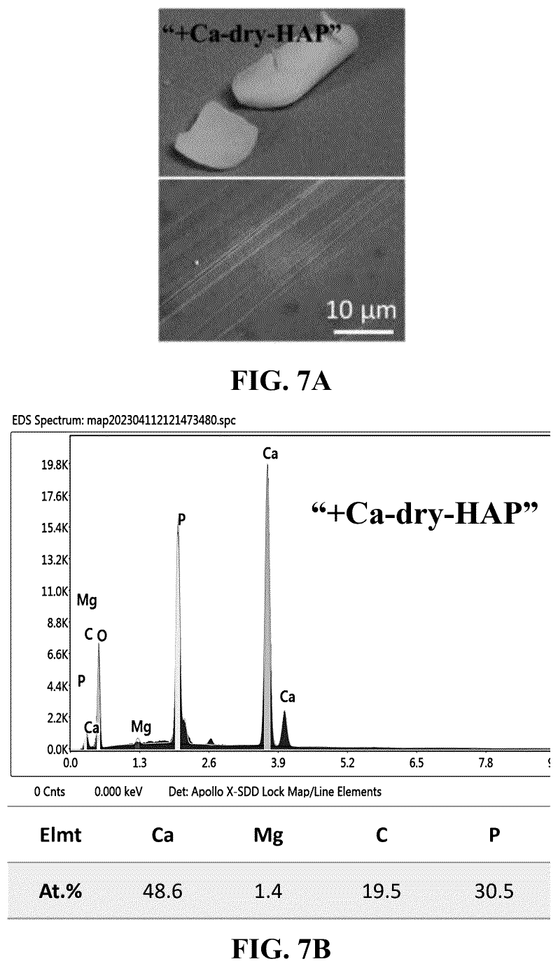

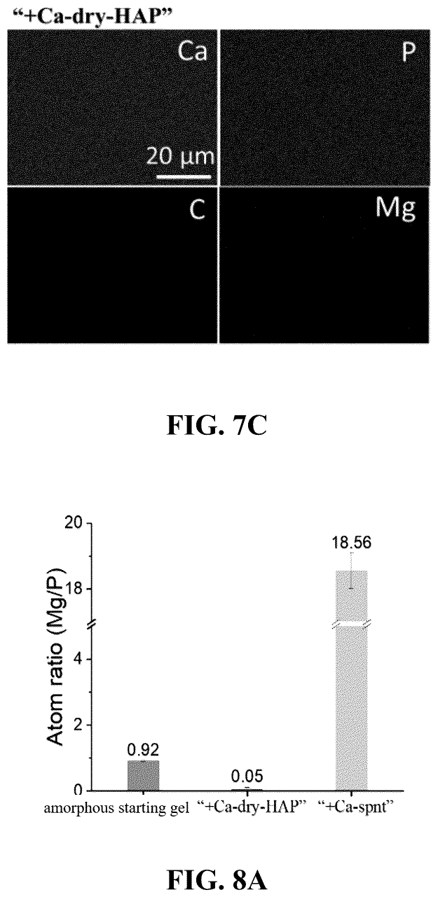

Crystalline Ca₂P₂O₇·2H₂O nanosheets emerged in the morphologically intact gel matrix shortly after the soaking treatment in the CaCl₂ solution, as revealed by the XRD and TEM examination (FIGS. 2C-2D, 4A-4B). Both EDX and ICP detected distinct compositions of the original stable and non-toxic amorphous starting gel and the air-dried product “+Ca-dry-HAP”. The atomic contents of Ca, Mg, C, and P were approximately equal for the former, but dramatically changed in “+Ca-dry-HAP”, showing only a trace amount of Mg, a considerably lowered C:P ratio (0.08:1 from the EDX results), and a boosted Ca:P ratio (1.83:1 from the ICP results) that was much closer to that of HAP (1.67:1) (FIGS. 5A-7C).

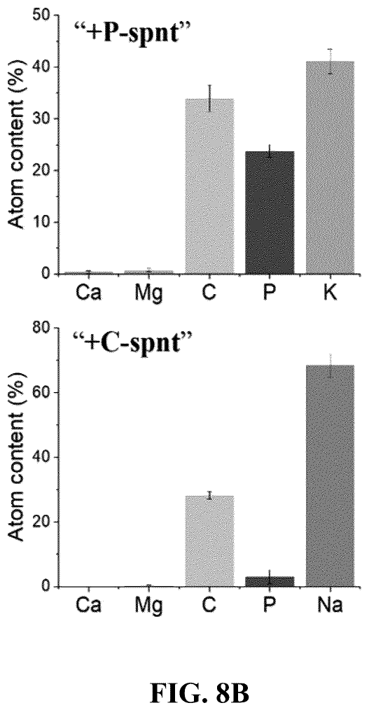

Moreover, the supernatant collected after the soaking reaction (denoted as “+Ca-spnt”) also contained a dramatically increased content of Mg and a lowered content of Ca (but little change for the contents of C, P, and O), indicating the replacement of Mg²⁺ in the stable and non-toxic amorphous starting gel by Ca²⁺ from the soaking solution (FIG. 8). Perhaps, the lattice distortion and stress induced by the replacement of the smaller Mg²⁺ ions with the bigger Ca²⁺ ions greatly facilitated the condensation reaction of phosphates to produce the unusual mineral of pyrophosphate. The phosphate condensation, in turn, relieved the lattice stress and allows the exchange of Mg²⁺ by Ca²⁺ to proceed throughout the entire mineral component of the gel. Finally, calcium pyrophosphate and ACC were produced as a result of the ion exchange and phosphate condensation reactions.

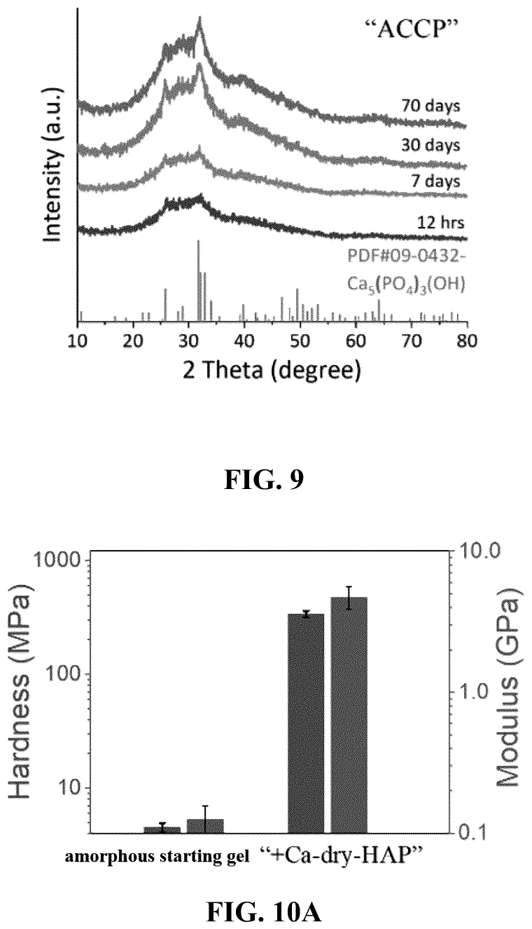

By comparison, the ACCP gel spontaneously transformed into HAP without forming the intermediate of CPP (FIG. 9), indicating the kinetic ion replacement of Mg²⁺ by Ca²⁺ in the stable and non-toxic amorphous starting gel to be crucial for the formation of pyrophosphate. It should also be noted that the “ACCP” gel was more prone to crystallization than the stable and non-toxic amorphous gel. The as-made wet gel spontaneously produced small grains of HAP within hours under ambient conditions, and the crystallization process spanned at least seven days.

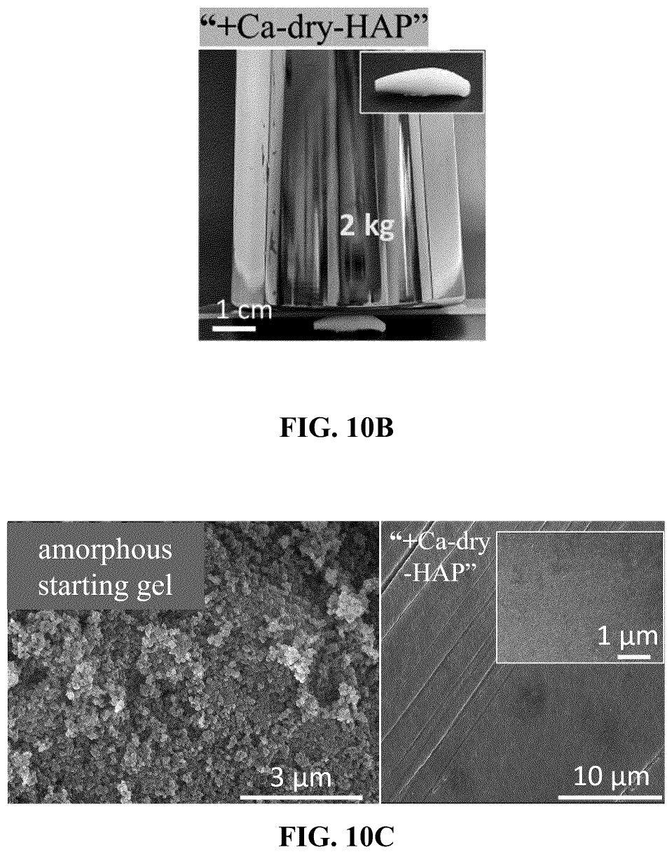

TEM study of “+Ca-dry-HAP” (ground and ultrasonically disbursed for TEM observation) revealed short nanorods whose SAED diffraction patterns exhibited the typical (002), (112), and (221) crystal planes of HAP, whereas FTIR measurement revealed peaks at 557 cm⁻¹ (ν₄ PO₄³⁻) and 1029 cm⁻¹ (ν₃ PO₄³⁻), indicating the formation of HAP in “+Ca-dry-HAP” (FIGS. 2C, 3 and 5A). Crystalline calcium pyrophosphate was observed in the freeze-dried product, whereas air-drying at 60° C. or room temperature led to bulky (e.g., 2 cm big) dense particles with considerable mechanical strength (modulus/hardness of 4.7/0.33 GPa, FIGS. 10A-10C).

The broad hump in the XRD pattern of “+Ca-frzdry-CPP” was consistent with the presence of ACC (FIG. 2C), and the generation of ample CO₂ bubbles when “+Ca-dry-HAP” was mixed with HCl indicates the existence of carbonate (FIG. 2E). These results together pointed to amorphous carbonate in “+Ca-dry-HAP”.

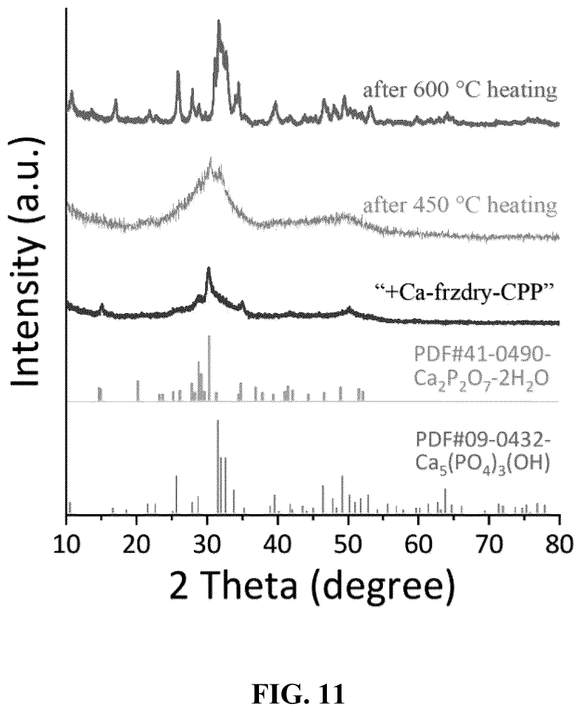

Reportedly, calcium phosphate, Ca₃(PO₄)₂, can be synthesized by heating CPP and calcite at high temperatures (e.g., 900-1300° C.)^{6,7}. However, in the present invention, at such a low temperature, CPP reacted with ACC to yield HAP in the air-dried “gel+Ca” (i.e., “+Ca-dry-HAP”). The lack of HAP formation in the freeze-dried “gel+Ca” (i.e., “+Ca-frzdry-CPP”) was also informative. Though air-drying at 60° C. led to HAP, thermal annealing of “+Ca-frzdry-CPP” did not generate HAP until the temperature was raised to 600° C. (FIG. 11), indicating that temperature alone did not determine the product profile.

The mild low-temperature synthesis of HAP achieved in the present invention was possibly due to the following factors that greatly lower the energy barrier of the reaction: 1) the 2D nanosheets of CPP produced here from the aqueous reaction under ambient conditions are more reactive than the conventionally prepared CPP powder; 2) the amorphous, hydrated, and colloidal nature of ACC is more reactive than the calcite powder which is crystalline, anhydrous, and of large particle sizes (e.g., it is very likely that the dehydration of ACC disrupts its structural stability and enhances its reactivity); 3) the aqueous reaction environment allows a large reactive interface between ACC and 2D CPP; 4) the uneven structural shrinkage and rearrangement during the gradually progressed dehydration under mild conditions builds up high internal stress to tighten the entangled ACC/2D CPP and promote their reaction.

### Preparation of the “Gel+P”, “+P-Spnt”, and “+P-Dry-HAP” Samples

The wet stable and non-toxic amorphous starting gel (15 g) from Example 2 was dispersed into 30 ml solution of K₂HPO₄ (0.4, 0.6, 0.8, 1.0, 1.5, or 13.44 M) under the Vortex agitation. After standing for 15 hours, the white suspension was centrifuged. The supernatant collected from the sample treated with 13.44 M K₂HPO₄ solution was collected and labeled as “+P-spnt”. The precipitates were washed three times (by being redispersed in DI water, centrifuged, and decanted) to remove the soluble ions. After draining the supernatant, the precipitate thus obtained was a gelatinous substance, named as “gel+P”. The product dried at 60° C. was denoted as “+P-dry-HAP”.

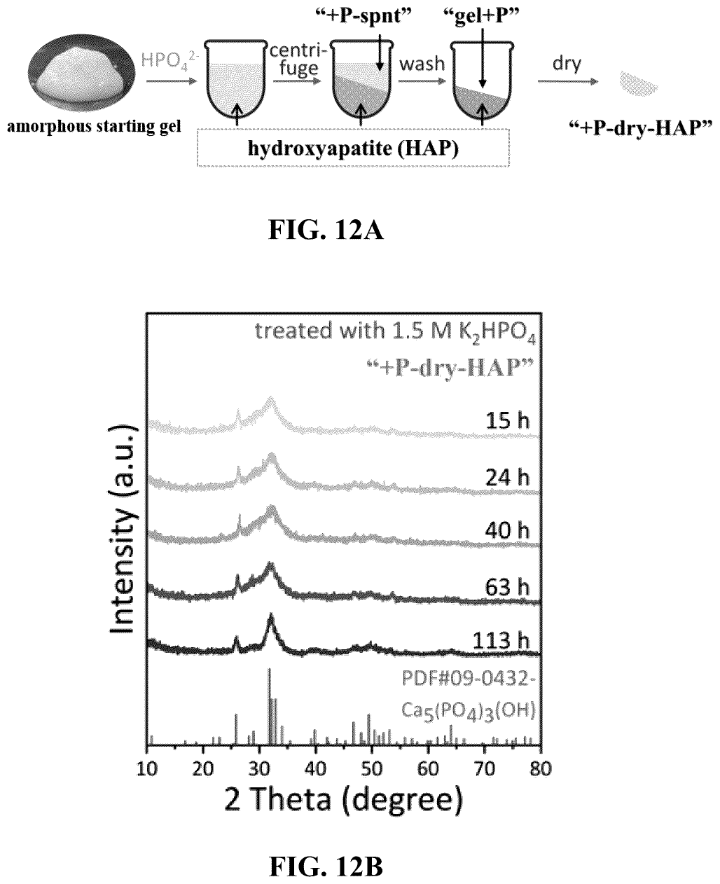

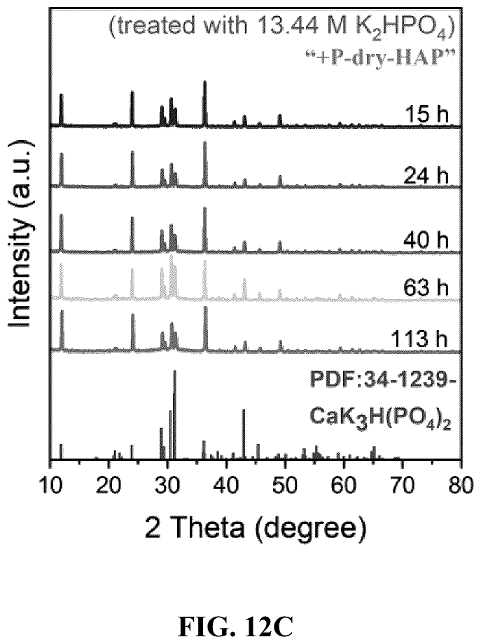

Crystalline CaK₃H(PO₄)₂ was resulted from immersion in a saturated (e.g., 13.4 M) solution of K₂HPO₄, whereas a dilute (e.g., 0.4 or 1.5 M) K₂HPO₄ soaking solution led to HAP (FIGS. 12A-12C). Unlike what was observed for the treatment with CaCl₂), no intermediate product of pyrophosphate was observed, and no drying procedure was needed for producing HAP. The carbonate ions of the stable and non-toxic amorphous starting gel were substituted by the phosphate ions in the K₂HPO₄ solution, leading to HAP and CaK₃H(PO₄)₂ in diluted and condensed soaking solutions, respectively:

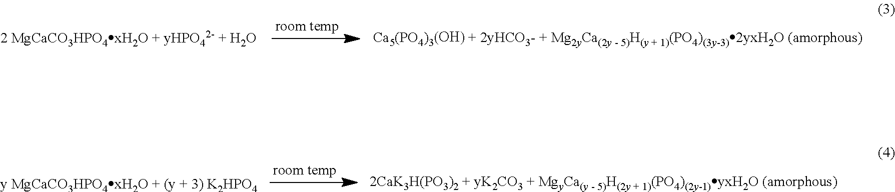

### Preparation of “Gel+C”, “+C-Spnt”, “+C-Dry-CaC” Samples

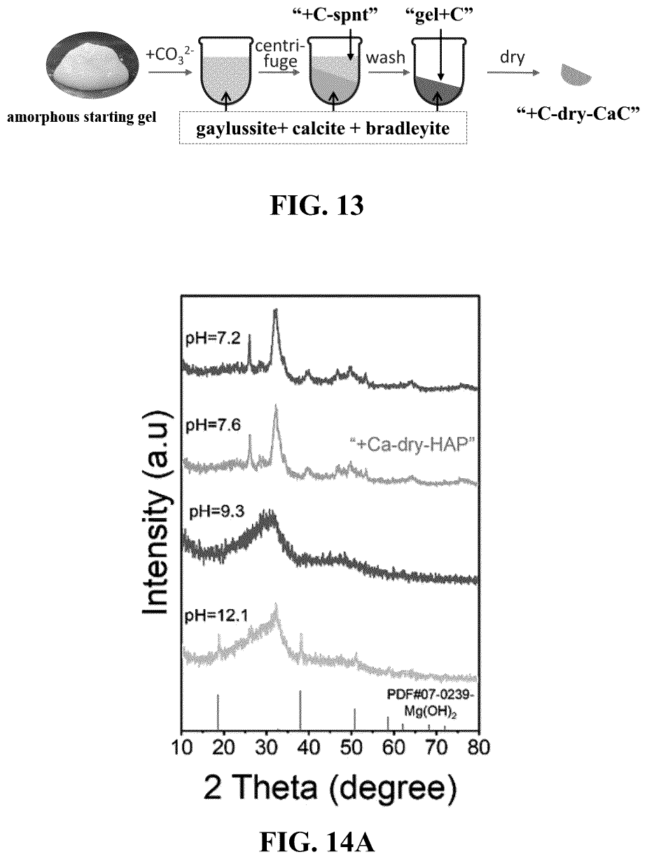

Referring to FIG. 13, the wet stable and non-toxic amorphous starting gel (15 g) from Example 2 was dispersed into 30 ml solution of Na₂CO₃ (0.4, 0.6, 0.8, 1.0, 1.5, 1.93 M) under Vortex agitation. After standing for 15 hours, the white suspension was centrifuged. The supernatant collected from the sample treated with 1.93 M Na₂CO₃ was collected and labeled as “+C-spnt”. The precipitates were washed three times (by being redispersed in DI water, centrifuged, and decanted) to remove the soluble ions. After draining the supernatant, the precipitate thus obtained was a gelatinous substance, named as “gel+C”. The product dried at 60° C. was denoted as “+C-dry-CaC”.

EDX measurements indicated the release of phosphate ions from the stable and non-toxic amorphous starting gel during the initial soaking process, while XRD analysis suggested the formation of calcite. Upon prolonging the soaking time in a concentrated Na₂CO₃ solution, additional crystals were generated from the stable and non-toxic amorphous starting gel, such as Na₂Ca(CO₃)₂·5H₂O, and Na₃Mg(PO₄)(CO₃):

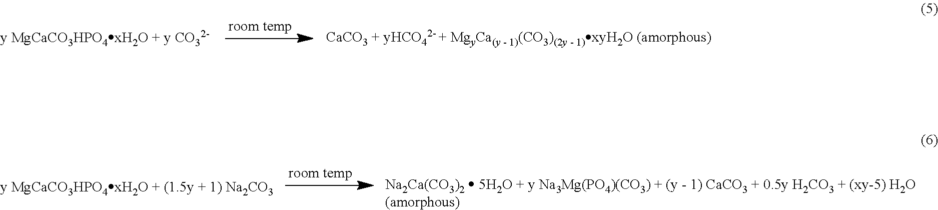

### Example 4

### Effect of pH Value on the Ion-Triggered Transformation

The pH value of the soaking environment was found to significantly impact the ion-triggered transformation, as demonstrated by the mineralization reactions in three sets of soaking solutions: 1) CaCl₂ solutions with varying pH values; 2) solutions containing KH₂PO₄, K₂HPO₄, and K₃PO₄; and 3) solutions containing NaHCO₃ and Na₂CO₃.

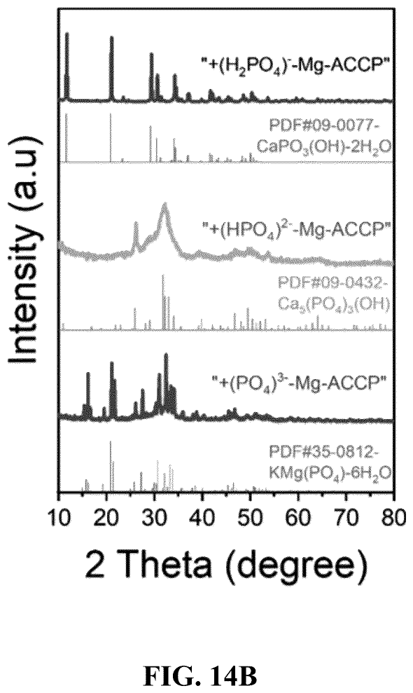

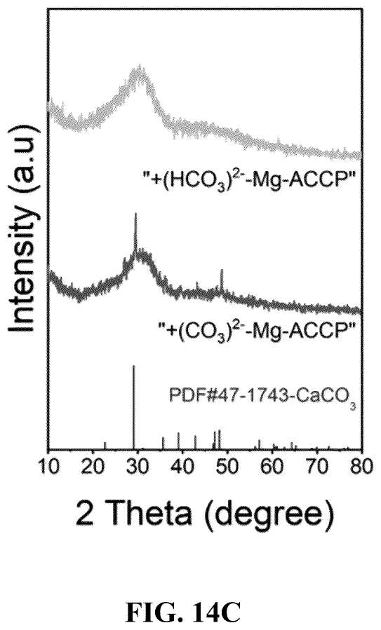

For the CaCl₂ solutions, a neutral pH value leaded to the production of HAP, a moderately alkaline solution hindered the mineralization, and a more alkaline environment resulted in crystalline Mg(OH)₂ (FIG. 14A). For the phosphate solutions, KH₂PO₄, K₂HPO₄ and K₃PO₄ (each of 1 M, pH values of 8.7, 5.2, and 12.7, respectively) produced CaHPO₄·2H₂O, HAP, KMgPO₄·6H₂O (FIG. 14B). For the carbonate solutions, mineralization product of calcite was enabled with Na₂CO₃ (1 M, pH of 9.7) but not NaHCO₃ (1 M, pH of 11.5) (FIG. 14C).

### Example 5

### Application of the Stable and Non-Toxic Amorphous Starting Gels on Osteogenesis

The stable and non-toxic amorphous starting gel of the present invention was fully inorganic, based on biocompatible ions (such as Ca²⁺, Mg²⁺, CO₃²⁻, and PO₄²⁻) common in bone marrow mesenchymal stem cells.

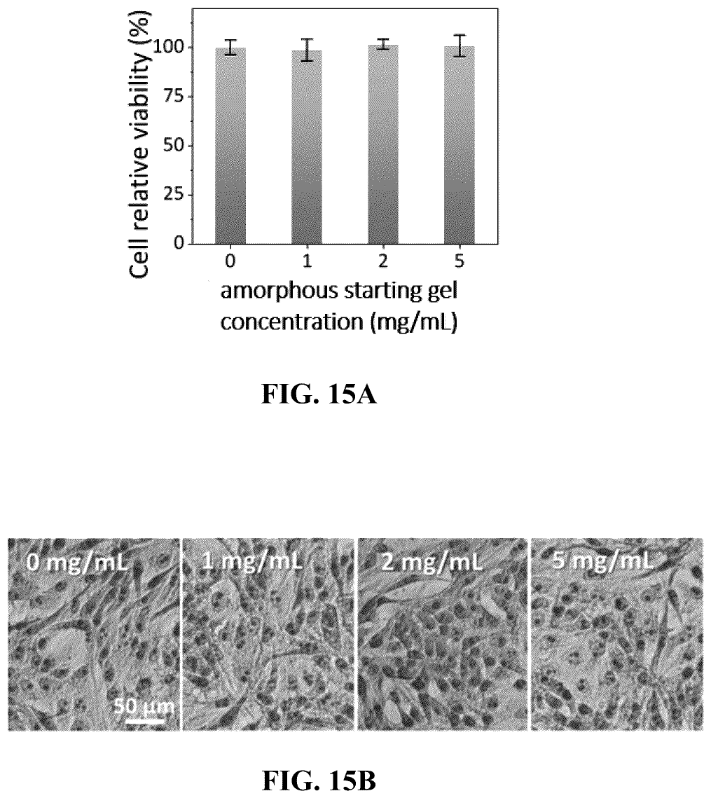

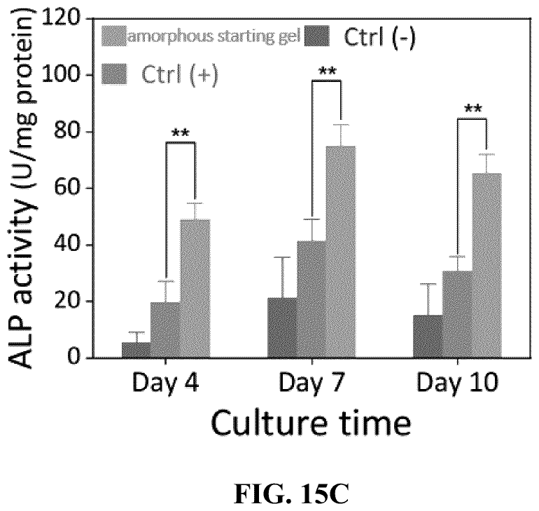

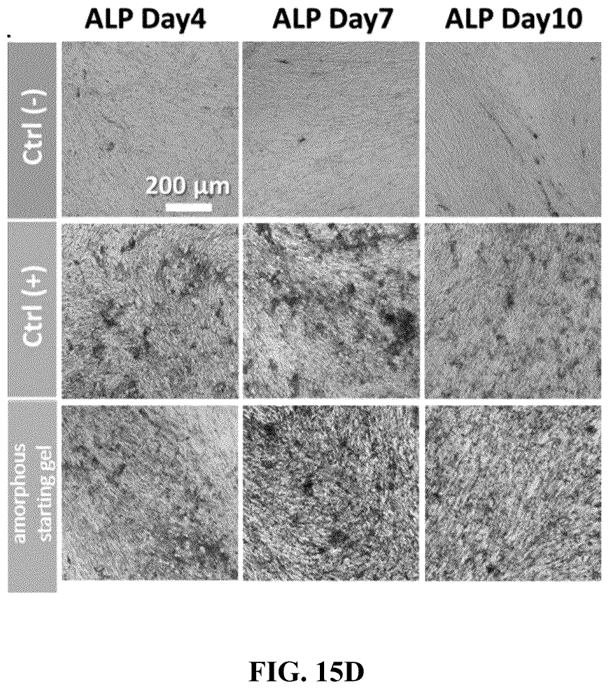

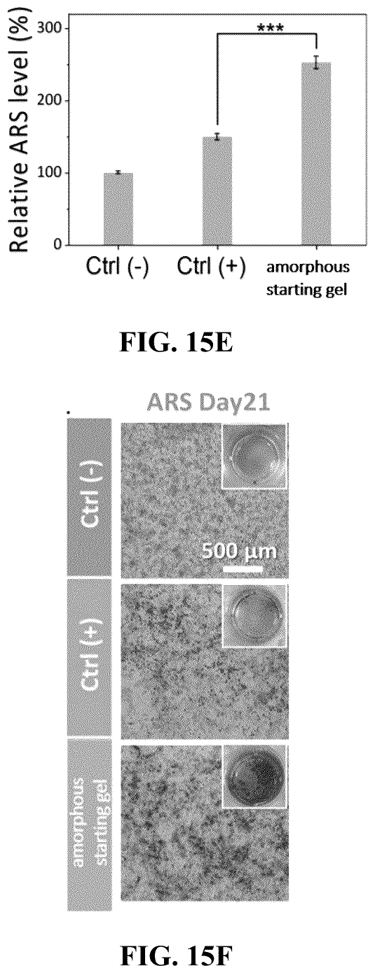

Cytotoxicity assay and histological staining images revealed the outstanding biocompatibility of the gel (cell relative viability as high as 100.8% at 5 mg/mL, FIG. 15A), which did not affect the proliferation and morphology of the BMSCs (FIG. 15B).

The ALP assay was further utilized to investigate its osteoinductivity (FIGS. 15C-15D). Compared with the positive control, the stable and non-toxic amorphous starting gel (5 mg/mL) delivered a significantly higher value as early as Day 4, while the osteoinductivity was further strengthened over time with the ALP activity value reaching 65.33 U/mg protein on Day 10, which was more than two times higher than the positive control group (30.75 U/mg protein). The ALP assay results illustrated the excellent osteogenic differentiation properties of the stable and non-toxic amorphous starting gel.

The osteogenesis capability of stable and non-toxic amorphous starting gel was examined by ARS staining (FIGS. 15E-15F). After 21 days of culturing, both the positive control and the stable and non-toxic amorphous starting gel group (5 mg/mL) exhibited the biomineralization phenomenon of calcium deposition. However, more calcium nodules were formed in the latter, as evidenced by its clearly higher ARS staining intensities. This finding was consistent with the quantitative values of the ARS absorbance (FIG. 15E), which found a 1.7 times higher absorbance value compared to the positive control.

These observations confirmed the remarkable ability of the stable and non-toxic amorphous starting gel to enhance bone growth under the physiological conditions with abundant Ca²⁺ in the environment, suggesting its potential as an ideal candidate for orthopedic applications.

### Example 6

### Application of the Synthesized Biominerals on Tooth Enamel Repairing

It is known that the outermost and hardest mineral layer of human teeth, enamel, contains over 95 wt % of HAP. Teeth sensitivity caused by enamel damage is a prevalent condition affecting many people, and there is an urgent need for safe and gentle techniques to repair enamel and to mitigate tooth sensitivity.

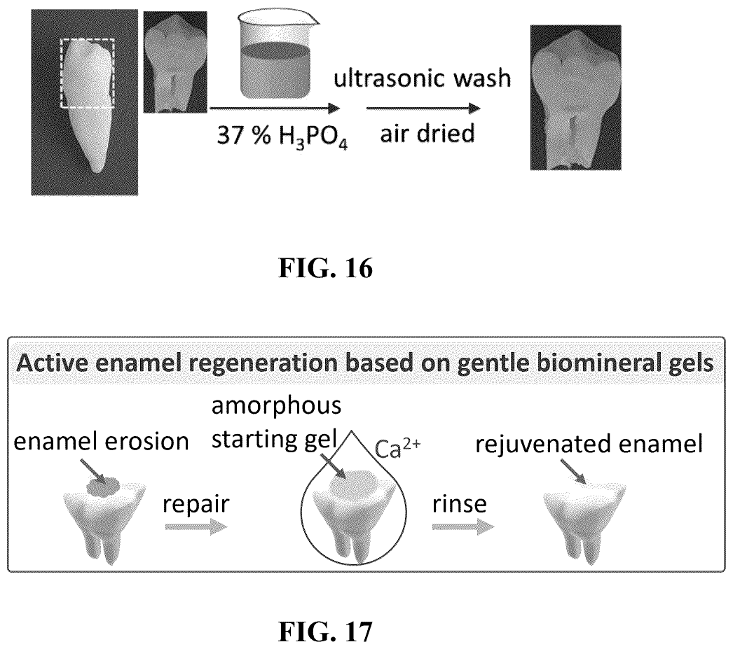

To imitate teeth with worn enamel, native teeth were acid-etched to expose the mineral structures of HAP. The native teeth were washed with DI water and ethanol each 3 times and dried in air. To mimic natural erosion, the teeth were soaked in H₃PO₄ solution (37%) for 10 sec followed by ultrasonic washing with DI water for 3 times and then dried in air (FIG. 16). The stable and non-toxic amorphous starting gels were smeared onto the damaged part, and then soaked in the CaCl₂ solution (6.7M), followed by the rinsing and drying treatment (FIG. 17). A piece of Si wafer with a weight of 200 g on top was placed onto the gel-coated part to enhance close contact between gel and the etched enamel. After approximately 10 hours, the wafer and weight were removed, and the enamel was air-dried in an oven at 60° C. for 1 hour. The treated tooth was finally thoroughly rinsed using DI water and air-dried at room temperature.

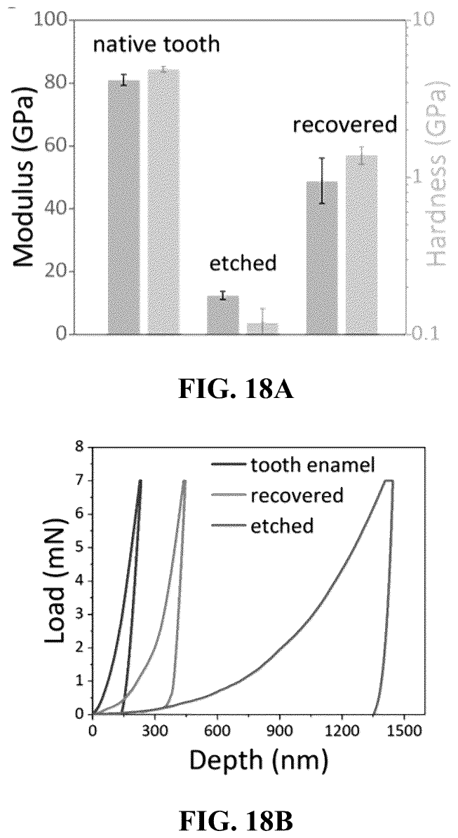

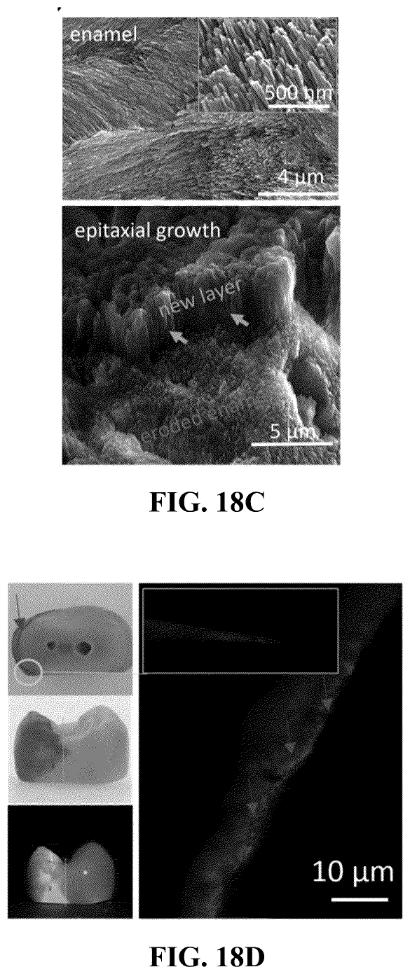

Epitaxial growth of HAP on the corroded enamel was achieved, giving rise to significant enhancement in mechanical strength (hardness/modulus restored from 0.1/12 to 1.4/49 GPa) (FIGS. 18A-18B).

The newly formed epitaxy layer was approximately 3 μm thick, characterized by densely packed HAP microfibers that were vertically aligned and seamlessly grew from the underlying enamel substrate.

In another test, calcein dye was employed to label the free Ca²⁺ ions in the soaking solution. Fluorescence images of the treated teeth revealed a distinct Ca-rich mineral coating, confirming the repair of enamel (FIGS. 18A, 18C-18D). This enamel-rejuvenation strategy is mild and convenient, requiring no harsh or toxic conditions, and holding promise in treating dental ailments, especially tooth sensitivity.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

The term “amorphous” describes a material that lacks a well-defined, orderly atomic structure. Unlike crystalline materials, which have atoms arranged in a repeating pattern, amorphous materials have a more random arrangement.

The term “ambient conditions” refer to the environmental conditions that are typical for the surrounding environment. For instance, the temperature often room temperature, typically ranging from about 15° C. to 25° C. The pressure is standard atmospheric pressure, which is approximately 1 atmosphere (atm). The relative humidity of the environment, which can vary but is usually around 30% to 50% in indoor settings.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### INDUSTRIAL APPLICABILITY

This invention will provide an applicable method for producing kinds of special minerals from a stable precursor and benefit for a large-scale production of inorganic mineral and health related materials at significantly reduced costs and simplified experimental operation. This advancement holds great potential in the fields of dentistry and bone regeneration, contributing to improved oral and skeletal health outcomes for individuals.

### REFERENCES: THE DISCLOSURES OF THE FOLLOWING REFERENCES ARE INCORPORATED BY REFERENCE

1. Boskey, A. L. Noncollagenous matrix proteins and their role in mineralization. Bone and Mineral 6, 111-123 (1989).
2. Hunter, G. K., Poitras, M. S., Underhill, T. M., Grynpas, M. D. & Goldberg, H. A. Induction of collagen mineralization by a bone sialoprotein-decorin chimeric protein. Journal of Biomedical Materials Research 55, 496-502 (2001).
3. Polishchuk, I. et al. Coherently aligned nanoparticles within a biogenic single crystal: a biological prestressing strategy. Science 358, 1294-1298 (2017).
4. Mu, Z. et al. Pressure-driven fusion of amorphous particles into integrated monoliths. Science 372, 1466-1470 (2021).
5. Du, H. et al. Additives: their influence on the humidity- and pressure-induced crystallization of amorphous CaCO₃. Chemistry of Materials 32, 4282-4291 (2020).
6. Kawano, J., Maeda, S. & Nagai, T. The effect of Mg²⁺ incorporation on the structure of calcium carbonate clusters: investigation by the anharmonic downward distortion following method. Physical chemistry chemical physics 18, 2690-2698 (2016).
7. Lei, C. et al. Biomimetic self-maturation mineralization system for enamel repair. Advanced Materials 36, 2311659 (2024).
8. Wei, Y. et al. Enamel repair with amorphous ceramics. Advanced Materials 32, 1907067 (2020).
9. Pérez-Castiñeira, J. R., Docampo, R., Ezawa, T. & Serrano, A. Editorial: pyrophosphates and polyphosphates in plants and microorganisms. Frontiers in Plant Science 12 (2021).

What is claimed is:

1. A method for synthesizing a calcium-based biomineral under ambient conditions, comprising:
mixing one or more biocompatible aqueous solutions containing more than three cationic species and/or anionic species to form a stable and non-toxic amorphous starting gel; and
transferring the stable and non-toxic amorphous starting gel into one or more salt solutions to trigger phase transformation to form the calcium-based biomineral,
wherein the one or more salt solutions are adopted in different concentrations.

2. The method of claim 1, further comprising washing and drying the stable and non-toxic amorphous starting gel.

3. The method of claim 1, wherein each of the one or more biocompatible aqueous solutions has a concentration ranging from 0.1 M to 1 M.

4. The method of claim 1, wherein the stable and non-toxic amorphous starting gel remains in an amorphous state and does not crystallize when exposed to temperatures below 600° C.

5. The method of claim 1, wherein the calcium-based biomineral comprises calcium orthophosphates, calcium carbonates, phosphate carbonates, or calcium pyrophosphates.

6. The method of claim 1, wherein the one or more biocompatible aqueous solutions comprise calcium chloride solution, magnesium chloride solution, sodium carbonate solution, dipotassium phosphate solution, or a combination thereof.

7. The method of claim 1, wherein the cationic species are selected from the group consisting of calcium, strontium, magnesium, manganese, iron, cobalt, nickel, copper, zinc, silver, gold, ammonium, sodium, potassium cations, and rare earth elements.

8. The method of claim 1, wherein the anionic species are selected from the group consisting of molybdate, tungstate, sulfate, silicate, phosphate, chloride, and carbonate anions.

9. The method of claim 1, wherein the one or more salt solution comprises CaCl₂ solution, K₂HPO₄ solution, or Na₂CO₃ solution.

10. The method of claim 1, wherein the stable and non-toxic amorphous starting gel is mixed with the one or more salt solution in a concentration ranging from 0.4 M to saturation.

11. The method of claim 1, wherein the calcium-based biomineral has an average particle size ranging from 1 nm to 700 nm.

12. A bone cement material comprising the calcium-based biomineral of claim 1.

13. A tooth repair material comprising the calcium-based biomineral of claim 1.
